(19) United States
(12) Patent Application Publication
(10) Pub. No.: US 2022/0178562 A1
(43) Pub. Date: Jun. 9, 2022

(54) MULTI-MOTOR AIR PURIFIER

(71) Applicant: Guangdong Cinotex Environmental Sci-Tech Co. Ltd., Zhongshan City (CN)

(72) Inventor: Haiyang LU, Shixing County (CN)

(21) Appl. No.: 17/136,868

(22) Filed: Dec. 29, 2020

(30) Foreign Application Priority Data
Dec. 8, 2020 (CN) 202022919275.9

(51) Int. Cl. F24F 7/007; F24F 3/16; A61L 9/22

(52) U.S. Cl. CPC F24F 7/007; A61L 2209/14; A61L 9/22; F24F 3/16

(57) ABSTRACT

This new practical patent has issued a type of multi-motor air purifier with chassis. The mentioned chassis possesses air inlet area and exhaust outlet area. A couple of motor positions are prepared for mounting the motor module. Furthermore, the mentioned motor module units are evenly aligned at the air inlet area. The single motor module or multi-motor motor module work together to inhale the airflow from the inlet area into the chassis, then exhaust it out of the outlet area. It possesses simple structure, compact arrangement and favorable design and more advantages. Therefore, the new air purifier is an outstanding product with excellent technical and economic performance.

### TECHNICAL FIELD

This new utility mainly refers to a kind of multi-motor air purifier.

### TECHNICAL BACKGROUND

Generally traditional air purifiers apply single motor modular structure. The wind pressure in the core part is especially strong yet the wind pressure in the surrounding area is very small This design brings about over inhalation in the center of the filter, thus failing in intercepting the formaldehyde, PM2.5 and other harmful substances. As a result, the hazardous matter flows out of the air outlet again to cause secondary pollution; In the meantime, the inhaling power at the surrounding part of filter is so weak that it cannot adequately inhale the formaldehyde, PM2.5 and else harmful substances; Otherwise, the rotating speed of single-motor is supposed to be very high to achieve the required blowing rate. This leads to large noise and increase of power consumption, which is not suitable for energy conservation and quietness.

### CONTENTS OF THE UTILITY MODEL

To solve the above issues, this utility model has presented a new structural solution. Following technical solutions are used in this multi-motor air purifiers:

This kind of multi-motor air purifier consists of a chassis. The chassis possesses air inlet area and air exhaust area. The air-inlet area has a couple of motor mounting positions to assemble the motor module. Furthermore, the motor module is evenly aligned in the air inlet area. The single-motor module or multi-motor module works together to inhale the air from inlet area into the chassis, then exhaust it out of the outlet area.

Preferably, the described motor module is mounted in the air exhaust area in a horizontal, vertical or circled style.

Preferably, the purifying module is placed between the air inlet area and the motor module. The purifying module is a filter plate which works to screen and intercept the particles in the airflow.

Preferably, the purifying module is an absorbent body that can produce static electric field.

Preferably, the purifying module is an adherent body that can produce negative ions. Preferably, the air inlet area is installed on the back of the chassis; and the air exhaust area is installed on the sides of the chassis.

Preferably, the motor module and the purifying module are mounted inside the chassis. Preferably, the motor module consists of the motor and the housing. The motor is installed inside the housing. The housing possesses air inlet and air exhaust outlet. The air inlet is connected to the air exhaust area; the exhaust outlet is connected to the air exhaust area as well.

Compared to the traditional technology, this new utility possesses following beneficial effects:

This patent technology applies multi-motor module. Its air inlet area and exhaust area are large. The size of motor module decreases to increase the air inlet area as much as possible. This design ensures even distribution of air pressure. That is, it prevents the great difference in wind pressure between the central part and the surrounding part, thus effectively improving the lifespan and the inhale capacity of filter by more than 35%. The new utility is favorable to reduce the usage of consumable material and even protect the environment. The rotating speed of the multi-motor module is lower than single motor unit. However, the multi-motor module can achieve the required blowing rate in less noise and less peak power consumption. This design contributes to energy conservation and quietness. Furthermore, the multi-point air exhaust area has increased by three times. Compared to the traditional air purifier, the noise of new utility has decreased by 40%. Accordingly, its power consumption has reduced to the lowest level with more than fifteen CADR per watt.

### DESCRIPTION OF FIGURES

FIG. 1 shows the structural diagram of the multi-motor air purifier in this new utility of multi-motor air purifier provided from relatively better cases.

FIG. 2 shows the partial structural diagram of the multi-motor air purifier in this new utility of multi-motor air purifier provided from relatively better cases.

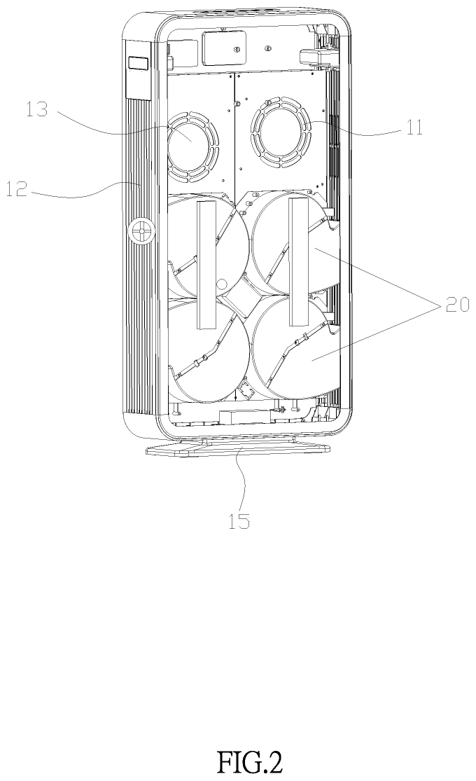

FIG. 3 shows the status diagram for the 1^{st} column of motor modules in this new utility of multi-motor air purifier provided from relatively better cases.

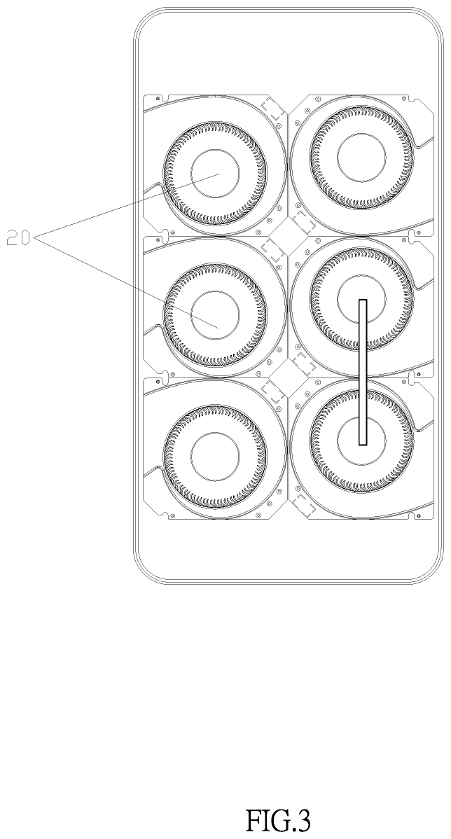

FIG. 4 shows the status diagram for the 2^{nd} column of motor modules in this new utility of multi-motor air purifier provided from relatively better cases.

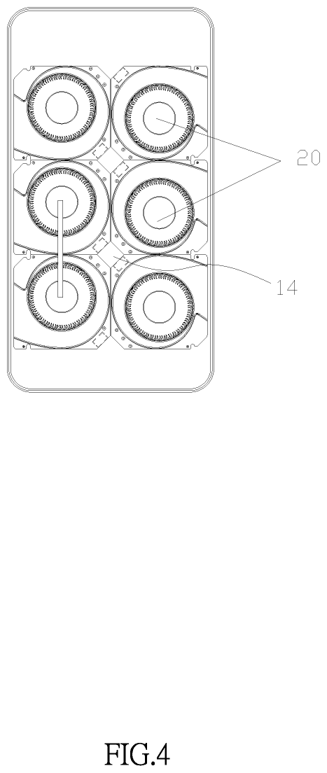

FIG. 5 shows the status diagram for the 3^{rd} column of motor modules in this new utility of multi-motor air purifier provided from relatively better cases.

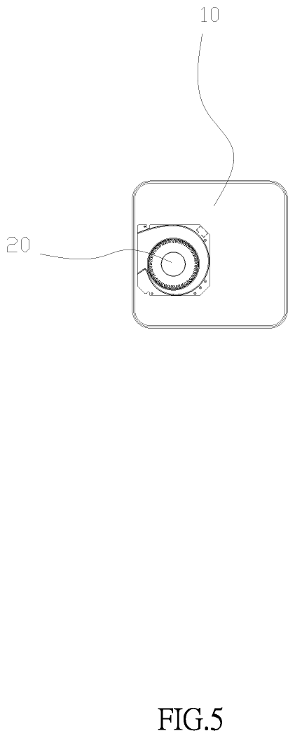

FIG. 6 shows the status diagram for the 4^{th} column of motor modules in this new utility of multi-motor air purifier provided from relatively better cases.

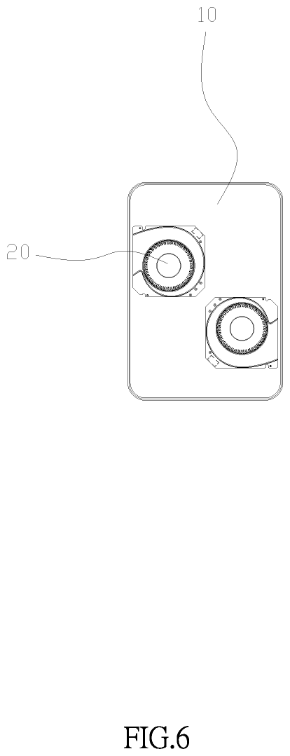

FIG. 7 shows the status diagram for the 5^{th} column of motor modules in this new utility of multi-motor air purifier provided from relatively better cases.

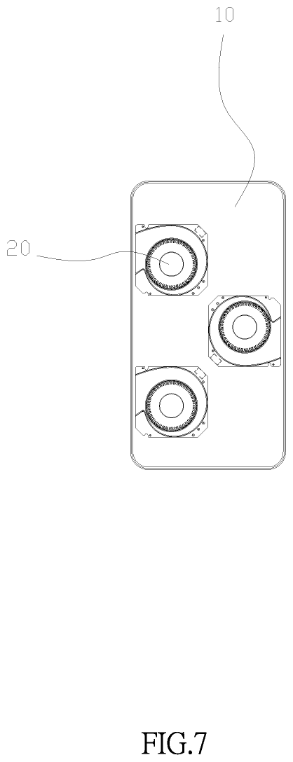

FIG. 8 shows the status diagram for the 6^{th} column of motor modules in this new utility of multi-motor air purifier provided from relatively better cases.

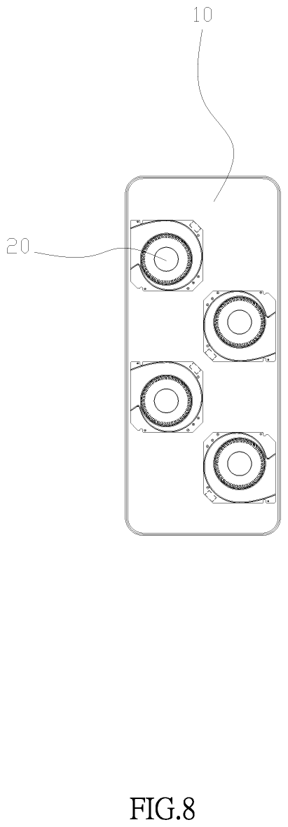

FIG. 9 shows the status diagram for the 7^{th} column of motor modules in this new utility of multi-motor air purifier provided from relatively better cases.

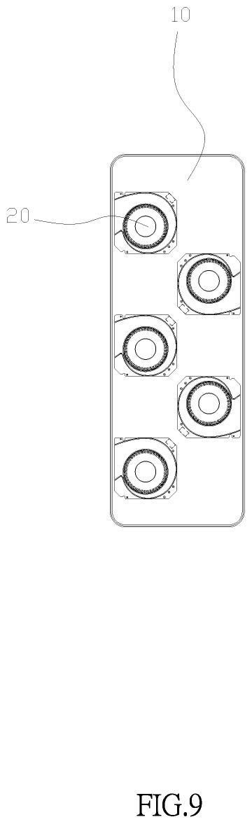

FIG. 10 shows the status diagram for the 8^{th} column of motor modules in this new utility of multi-motor air purifier provided from relatively better cases.

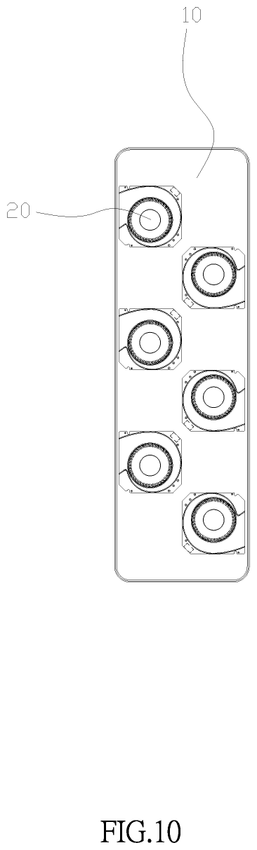

FIG. 11 shows the status diagram for the 9^{th} column of motor modules in this new utility of multi-motor air purifier provided from relatively better cases.

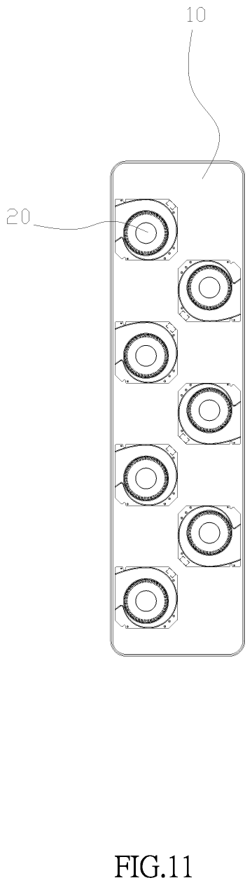

FIG. 12 shows the status diagram for the 10^{th} column of motor modules in this new utility of multi-motor air purifier provided from relatively better cases.

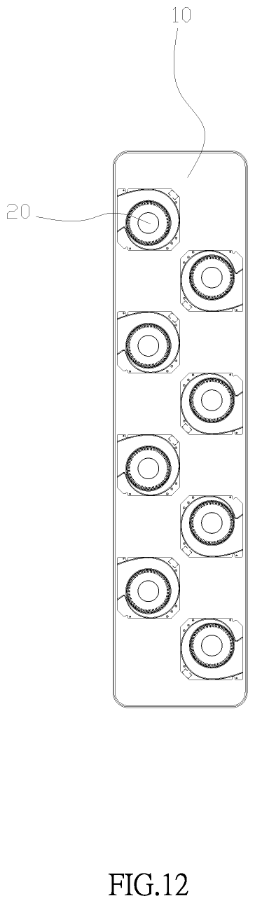

FIG. 13 shows the status diagram for the 11^{th} column of motor modules in this new utility of multi-motor air purifier provided from relatively better cases.

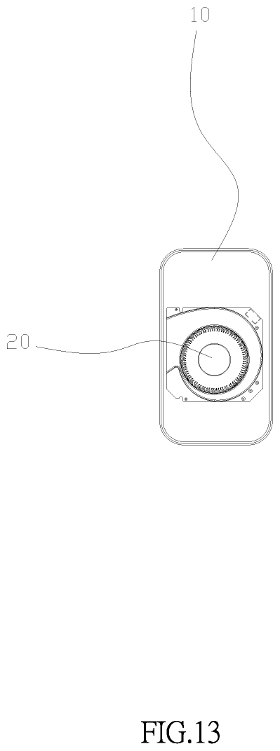

FIG. 14 shows the status diagram for the 12^{th} column of motor modules in this new utility of multi-motor air purifier provided from relatively better cases.

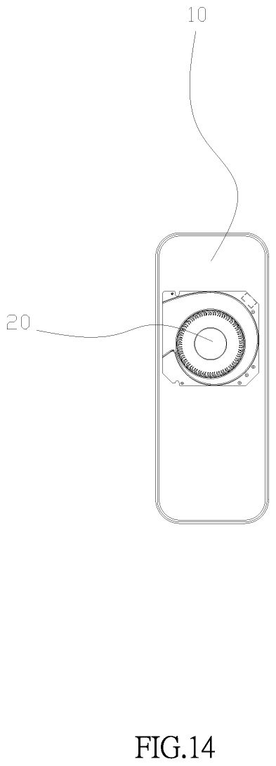

FIG. 15 shows the status diagram for the 13^{th} column of motor modules in this new utility of multi-motor air purifier provided from relatively better cases.

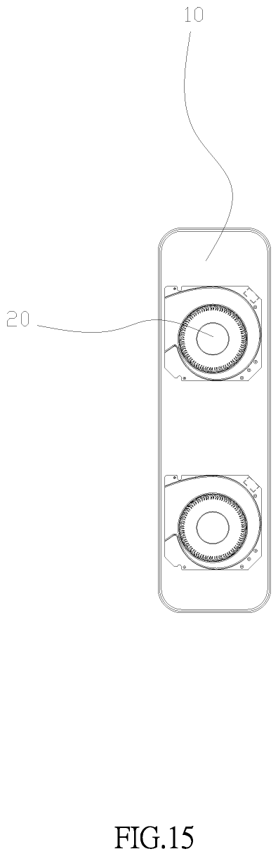

FIG. 16 shows the status diagram for the 14^{th} column of motor modules in this new utility of multi-motor air purifier provided from relatively better cases.

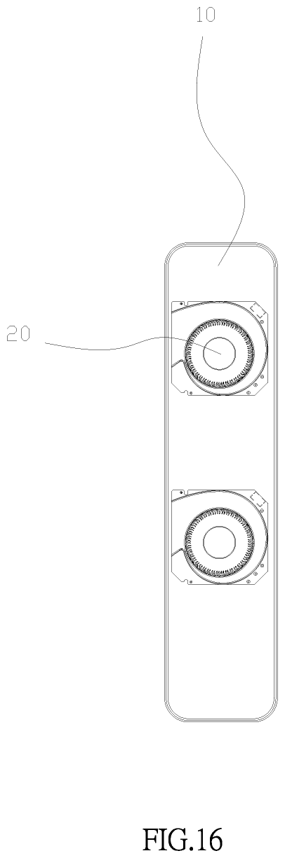

FIG. 17 shows the status diagram for the 15^{th} column of motor modules in this new utility of multi-motor air purifier provided from relatively better cases.

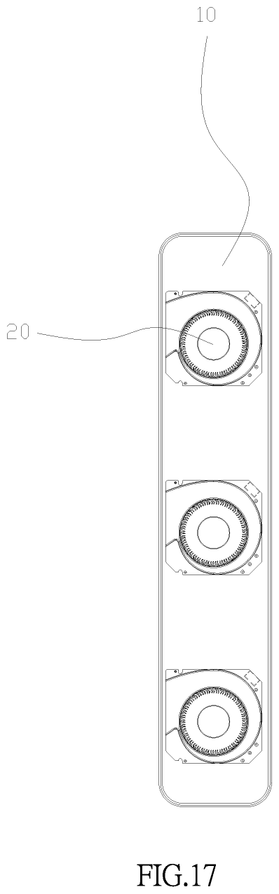

FIG. 18 shows the status diagram for the 16^{th} column of motor modules in this new utility of multi-motor air purifier provided from relatively better cases.

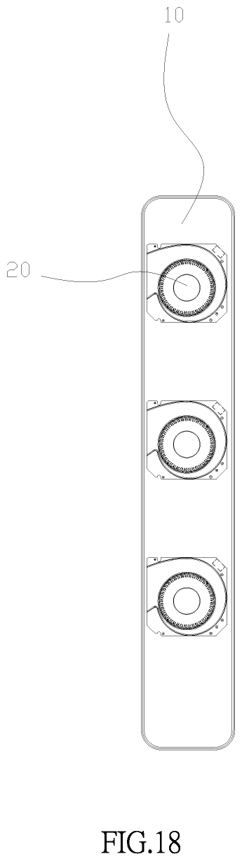

FIG. 19 shows the status diagram for the 17^{th} column of motor modules in this new utility of multi-motor air purifier provided from relatively better cases.

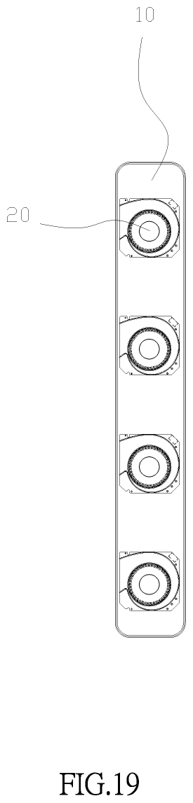

FIG. 20 shows the status diagram for the 18^{th} column of motor modules in this new utility of multi-motor air purifier provided from relatively better cases.

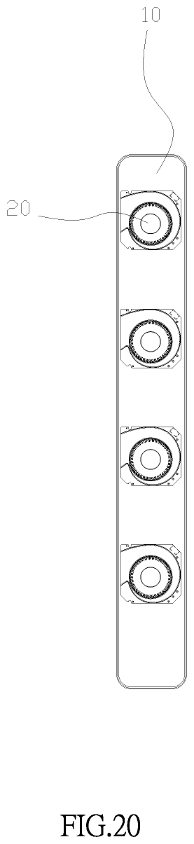

FIG. 21 shows the status diagram for the 19^{th} column of motor modules in this new utility of multi-motor air purifier provided from relatively better cases.

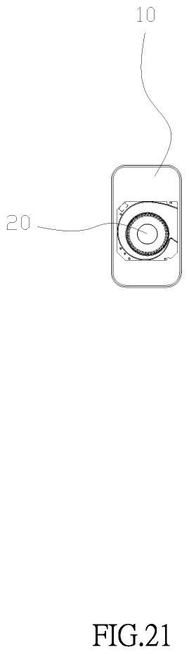

FIG. 22 shows the status diagram for the 20^{th} column of motor modules in this new utility of multi-motor air purifier provided from relatively better cases.

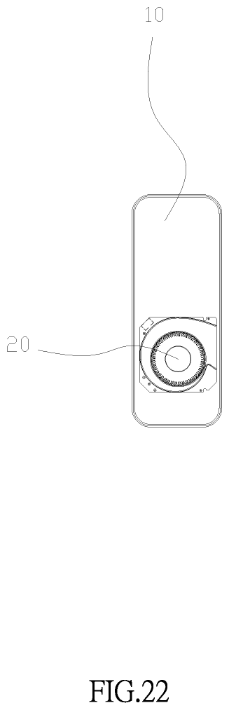

FIG. 23 shows the status diagram for the 21^{st} column of motor modules in this new utility of multi-motor air purifier provided from relatively better cases.

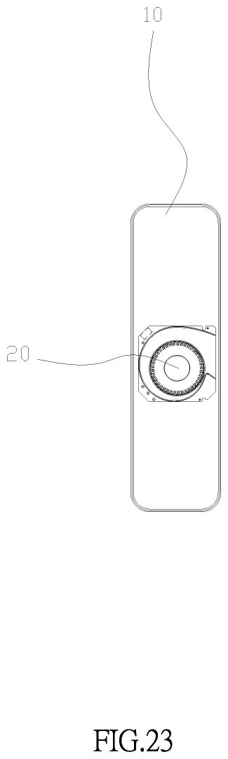

FIG. 24 shows the status diagram for the 22^{nd} column of motor modules in this new utility of multi-motor air purifier provided from relatively better cases.

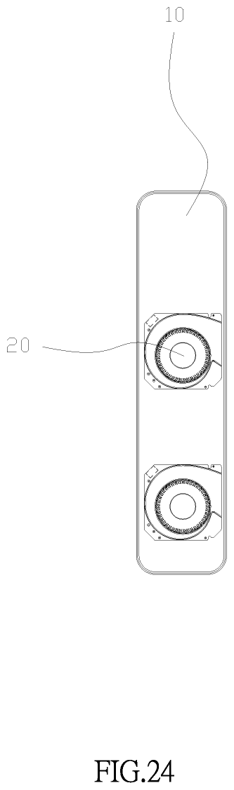

FIG. 25 shows the status diagram for the 23^{rd} column of motor modules in this new utility of multi-motor air purifier provided from relatively better cases.

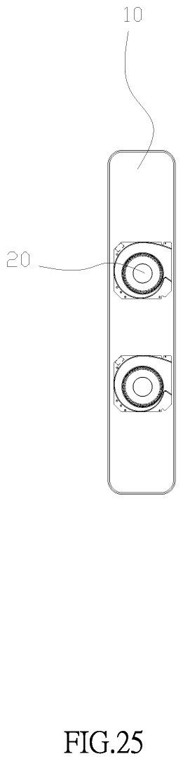

FIG. 26 shows the status diagram for the 24^{th} column of motor modules in this new utility of multi-motor air purifier provided from relatively better cases.

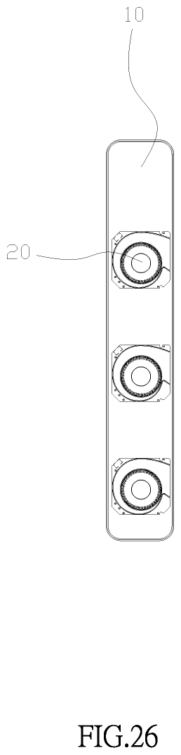

FIG. 27 shows the status diagram for the 25^{th} column of motor modules in this new utility of multi-motor air purifier provided from relatively better cases.

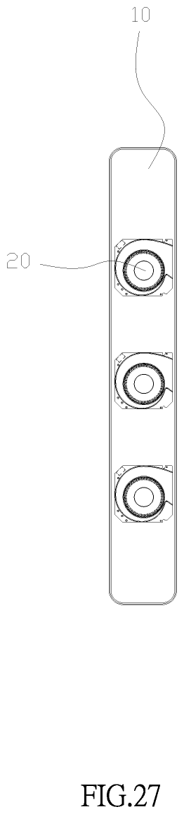

FIG. 28 shows the status diagram for the 26^{th} column of motor modules in this new utility of multi-motor air purifier provided from relatively better cases.

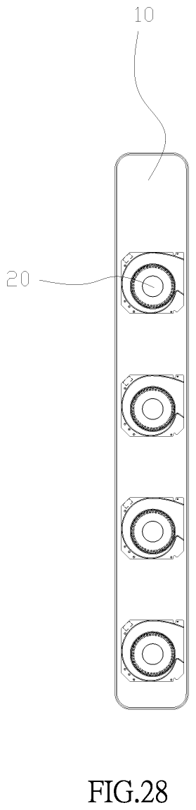

FIG. 29 shows the status diagram for the 27^{th} column of motor modules in this new utility of multi-motor air purifier provided from relatively better cases.

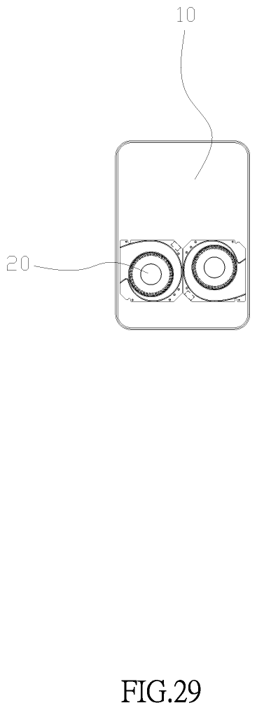

FIG. 30 shows the status diagram for the 28^{th} column of motor modules in this new utility of multi-motor air purifier provided from relatively better cases.

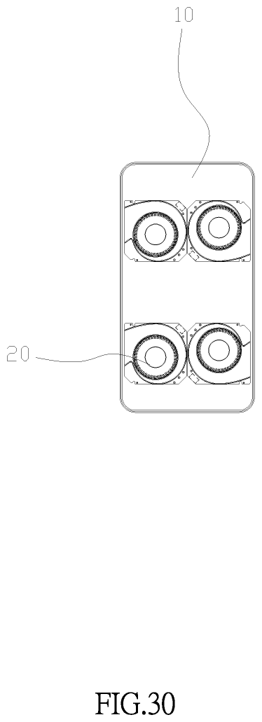

FIG. 31 shows the status diagram for the 29^{th} column of motor modules in this new utility of multi-motor air purifier provided from relatively better cases.

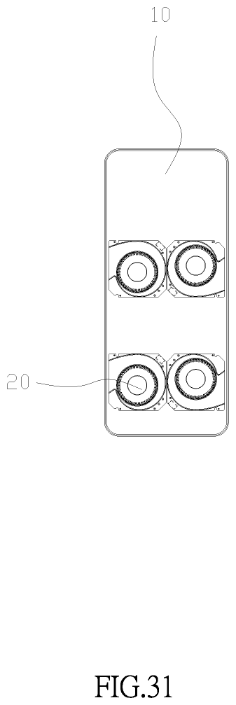

FIG. 32 shows the status diagram for the 30^{th} column of motor modules in this new utility of multi-motor air purifier provided from relatively better cases.

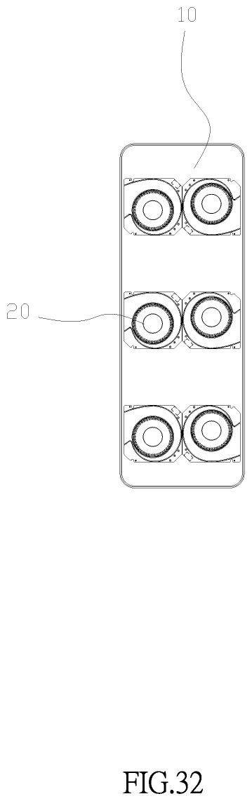

FIG. 33 shows the status diagram for the 31^{st} column of motor modules in this new utility of multi-motor air purifier provided from relatively better cases.

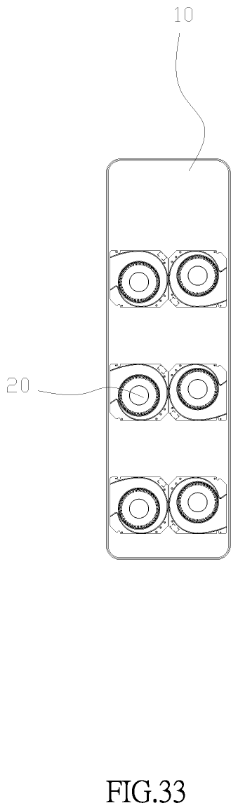

FIG. 34 shows the status diagram for the 32^{nd} column of motor modules in this new utility of multi-motor air purifier provided from relatively better cases.

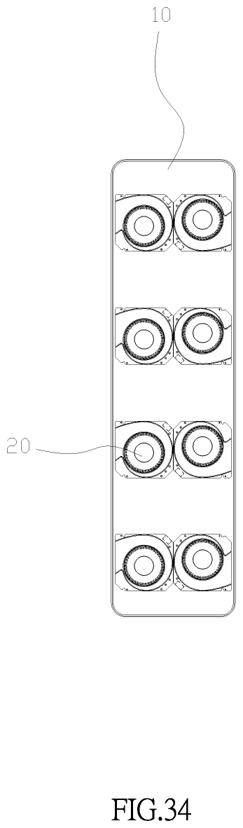

FIG. 35 shows the status diagram for the 33^{rd} column of motor modules in this new utility of multi-motor air purifier provided from relatively better cases.

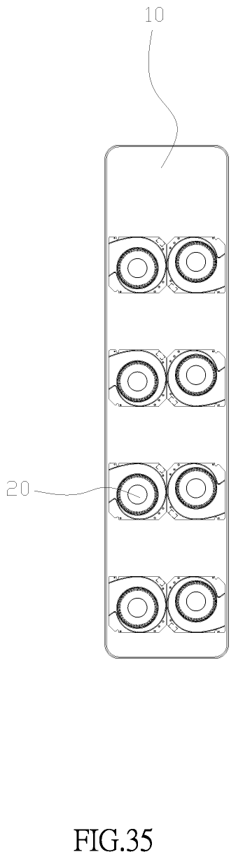

FIG. 36 shows the status diagram for the 34^{th} column of motor modules in this new utility of multi-motor air purifier provided from relatively better cases.

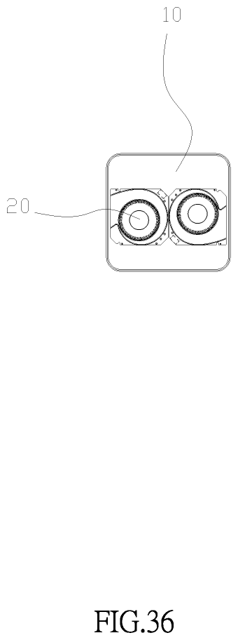

FIG. 37 shows the status diagram for the 35^{th} column of motor modules in this new utility of multi-motor air purifier provided from relatively better cases.

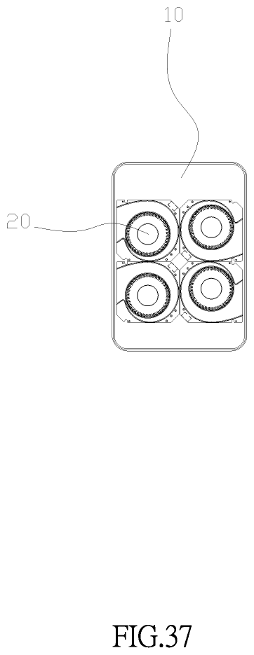

FIG. 38 shows the status diagram for the 36^{th} column of motor modules in this new utility of multi-motor air purifier provided from relatively better cases.

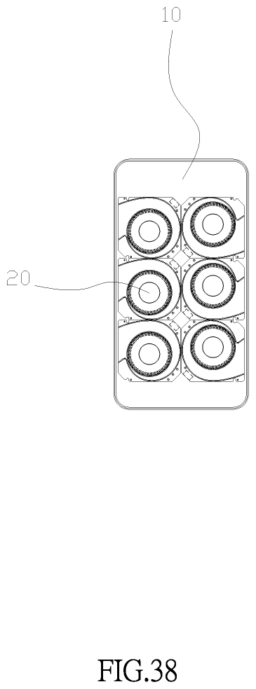

FIG. 39 shows the status diagram for the 37^{th} column of motor modules in this new utility of multi-motor air purifier provided from relatively better cases.

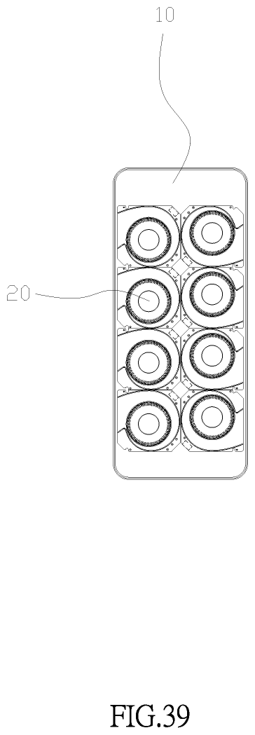

FIG. 40 shows the status diagram for the 38^{th} column of motor modules in this new utility of multi-motor air purifier provided from relatively better cases.

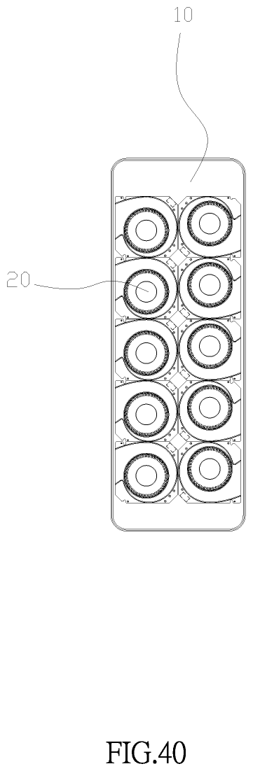

FIG. 41 shows the status diagram for the 39^{th} column of motor modules in this new utility of multi-motor air purifier provided from relatively better cases.

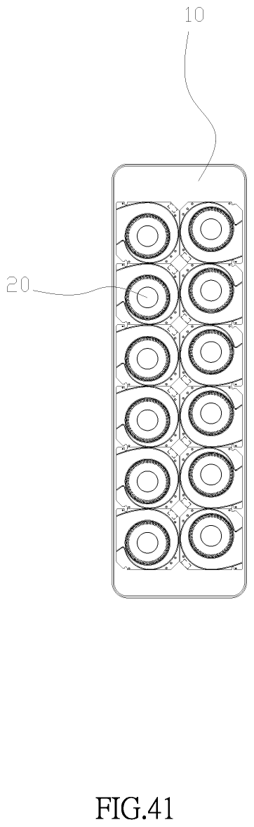

FIG. 42 shows the status diagram for the 40^{th} column of motor modules in this new utility of multi-motor air purifier provided from relatively better cases.

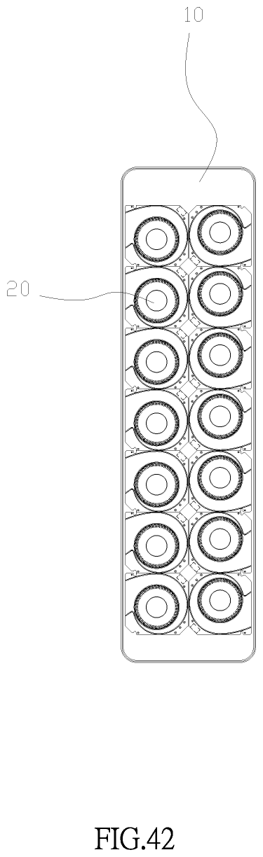

FIG. 43 shows the status diagram for the 41^{st} column of motor modules in this new utility of multi-motor air purifier provided from relatively better cases.

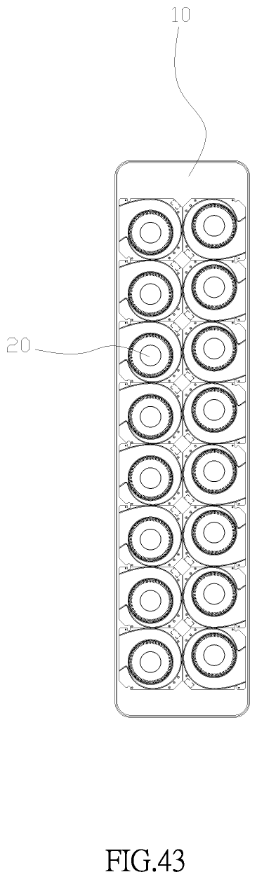

FIG. 44 shows the status diagram for the 42^{nd} column of motor modules in this new utility of multi-motor air purifier provided from relatively better cases.

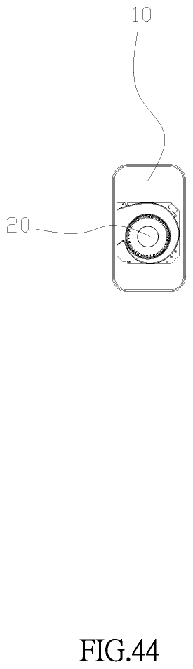

FIG. 45 shows the status diagram for the 43^{rd} column of motor modules in this new utility of multi-motor air purifier provided from relatively better cases.

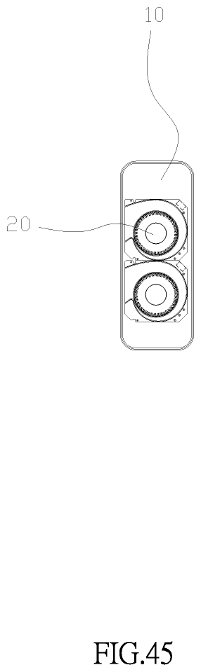

FIG. 46 shows the status diagram for the 44^{th} column of motor modules in this new utility of multi-motor air purifier provided from relatively better cases.

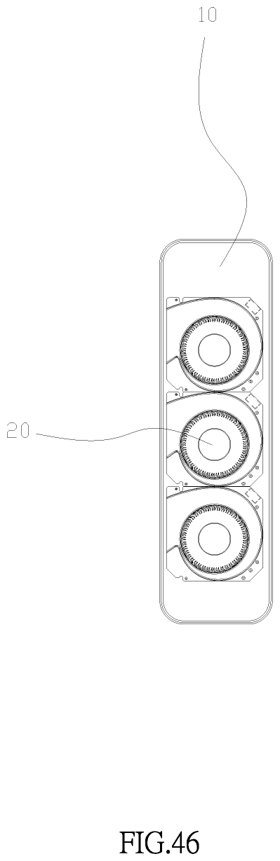

FIG. 47 shows the status diagram for the 45^{th} column of motor modules in this new utility of multi-motor air purifier provided from relatively better cases.

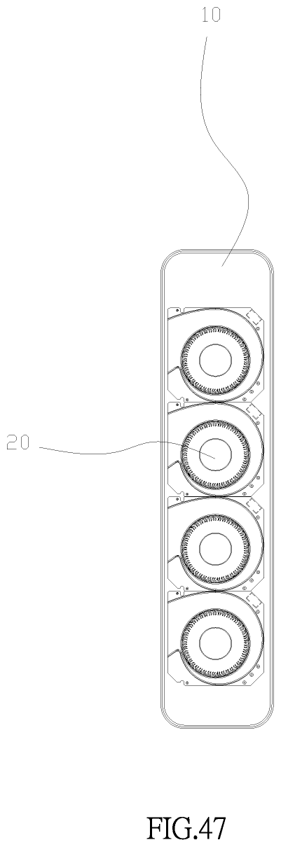

FIG. 48 shows the status diagram for the 46^{th} column of motor modules in this new utility of multi-motor air purifier provided from relatively better cases.

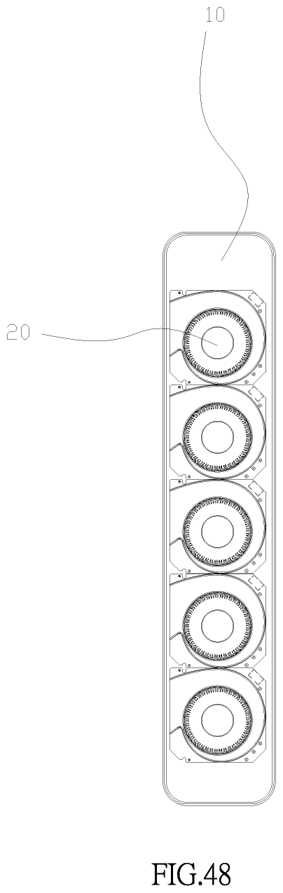

FIG. 49 shows the status diagram for the 47^{th} column of motor modules in this new utility of multi-motor air purifier provided from relatively better cases.

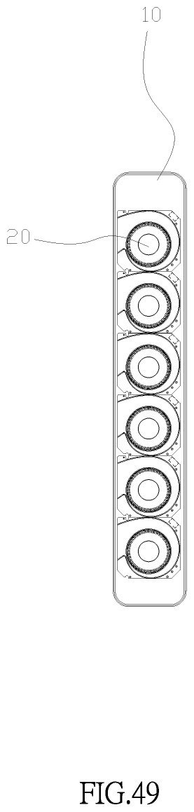

FIG. 50 shows the status diagram for the 48^{th} column of motor modules in this new utility of multi-motor air purifier provided from relatively better cases.

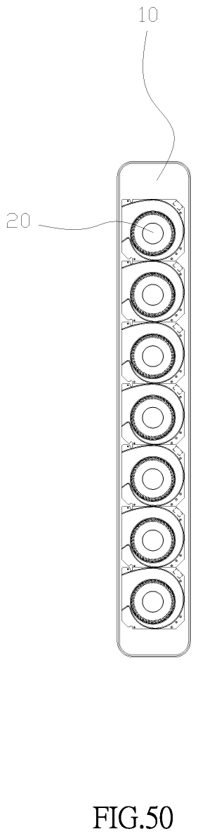

FIG. 51 shows the status diagram for the 49^{th} column of motor modules in this new utility of multi-motor air purifier provided from relatively better cases.

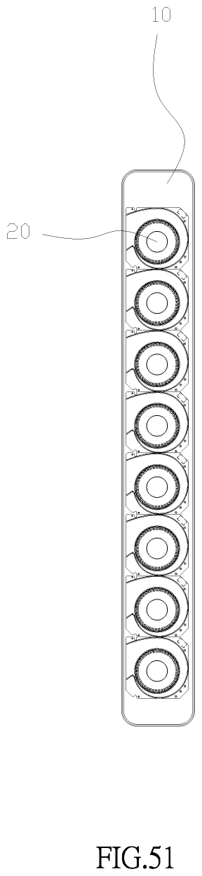

FIG. 52 shows the status diagram for the 50^{th} column of motor modules in this new utility of multi-motor air purifier provided from relatively better cases.

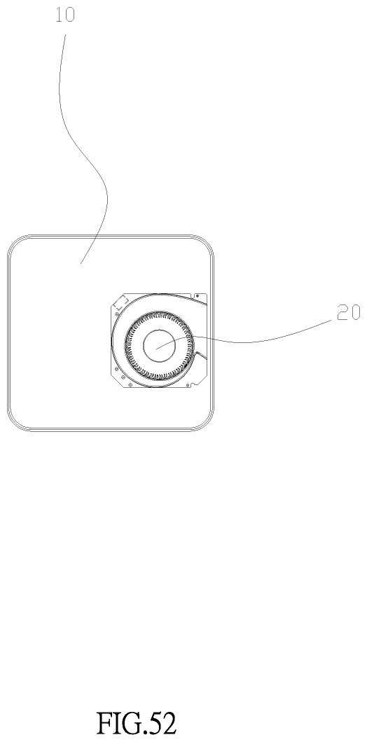

FIG. 53 shows the status diagram for the 51^{st} column of motor modules in this new utility of multi-motor air purifier provided from relatively better cases.

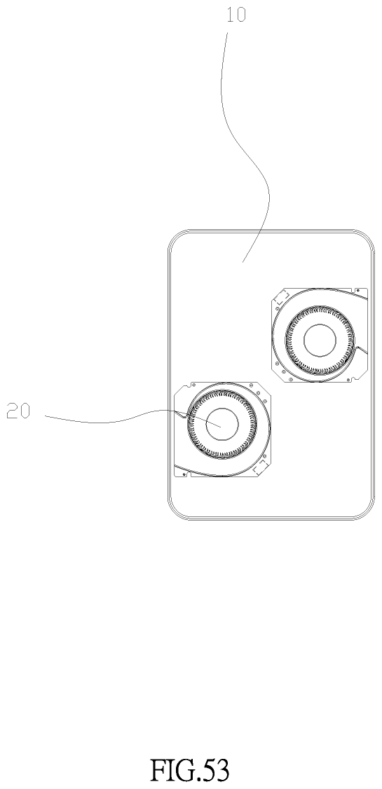

FIG. 54 shows the status diagram for the 52^{nd} column of motor modules in this new utility of multi-motor air purifier provided from relatively better cases.

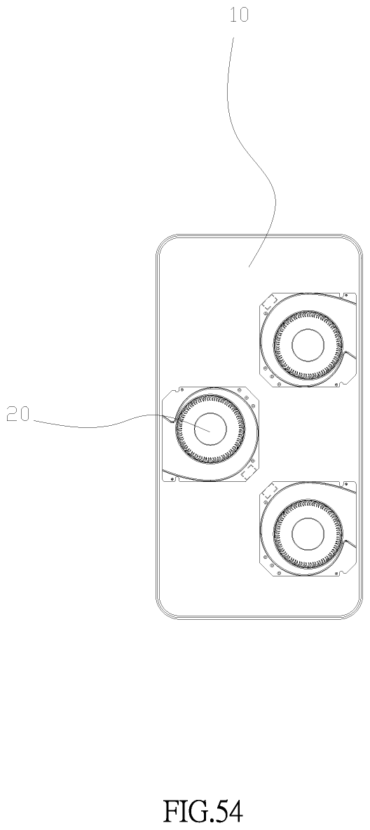

FIG. 55 shows the status diagram for the 53^{rd} column of motor modules in this new utility of multi-motor air purifier provided from relatively better cases.

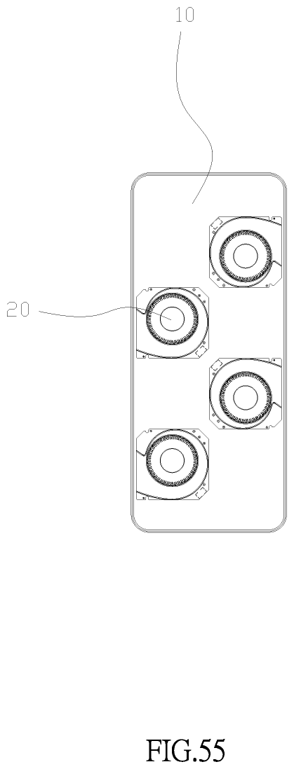

FIG. 56 shows the status diagram for the 54^{th} column of motor modules in this new utility of multi-motor air purifier provided from relatively better cases.

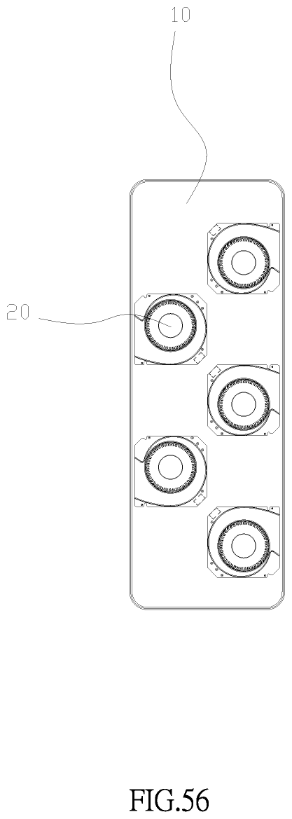

FIG. 57 shows the status diagram for the 55^{th} column of motor modules in this new utility of multi-motor air purifier provided from relatively better cases.

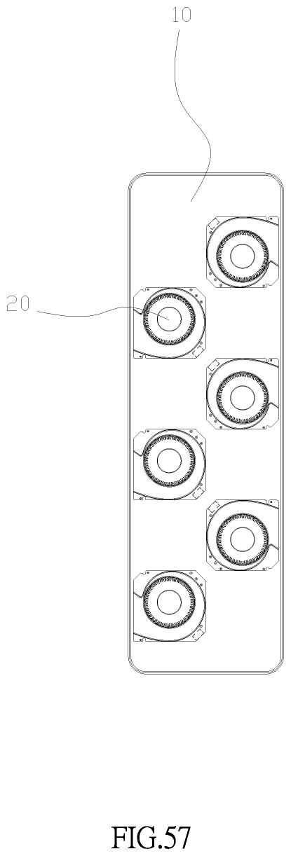

FIG. 58 shows the status diagram for the 56^{th} column of motor modules in this new utility of multi-motor air purifier provided from relatively better cases.

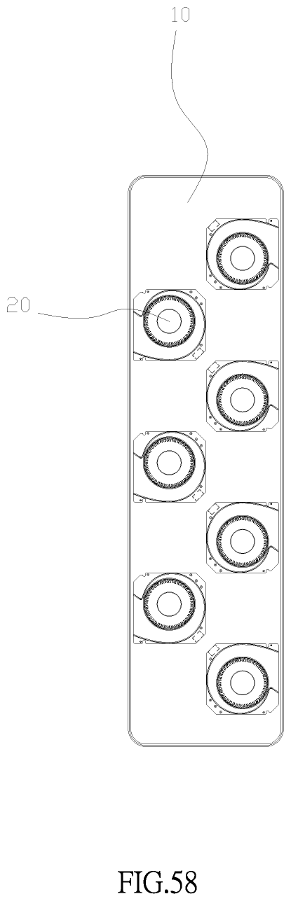

FIG. 59 shows the status diagram for the 57^{th} column of motor modules in this new utility of multi-motor air purifier provided from relatively better cases.

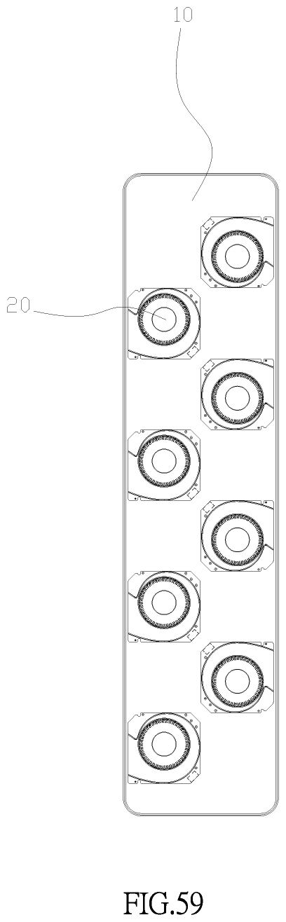

FIG. 60 shows the status diagram for the 58^{th} column of motor modules in this new utility of multi-motor air purifier provided from relatively better cases.

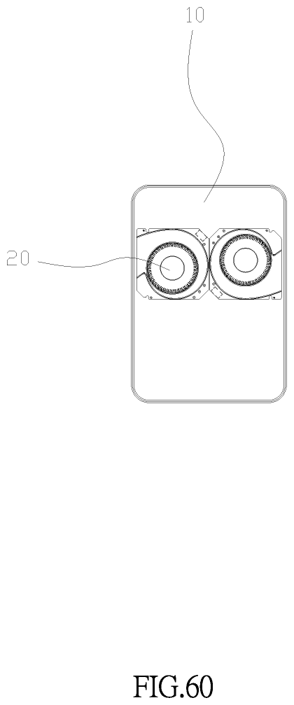

FIG. 61 shows the status diagram for the 59^{th} column of motor modules in this new utility of multi-motor air purifier provided from relatively better cases.

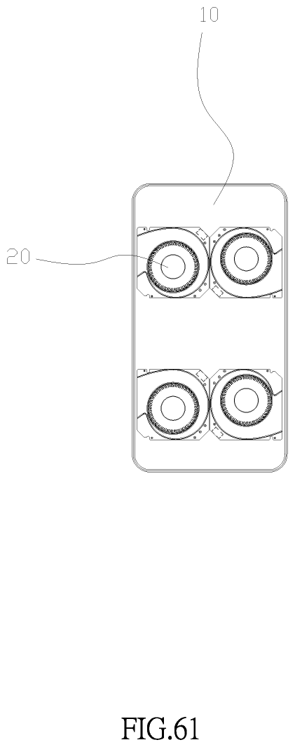

FIG. 62 shows the status diagram for the 60^{th} column of motor modules in this new utility of multi-motor air purifier provided from relatively better cases.

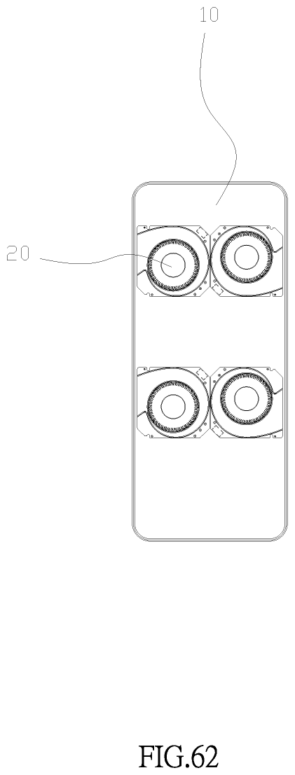

FIG. 63 shows the status diagram for the 61^{st} column of motor modules in this new utility of multi-motor air purifier provided from relatively better cases.

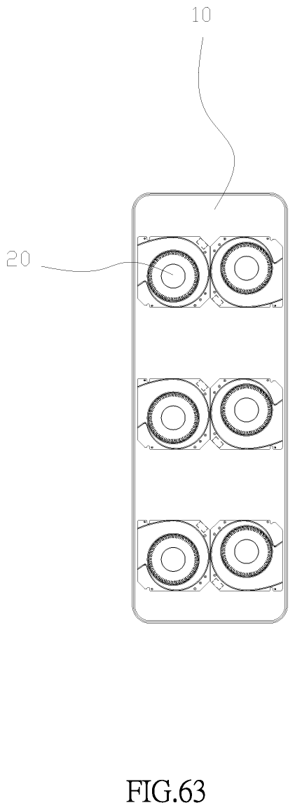

FIG. 64 shows the status diagram for the 62^{nd} column of motor modules in this new utility of multi-motor air purifier provided from relatively better cases.

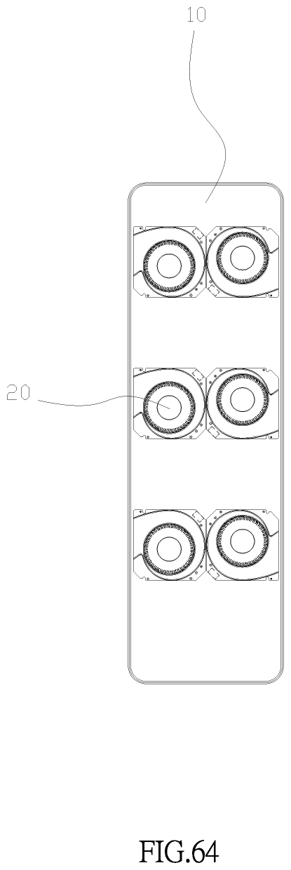

FIG. 65 shows the status diagram for the 63^{rd} column of motor modules in this new utility of multi-motor air purifier provided from relatively better cases.

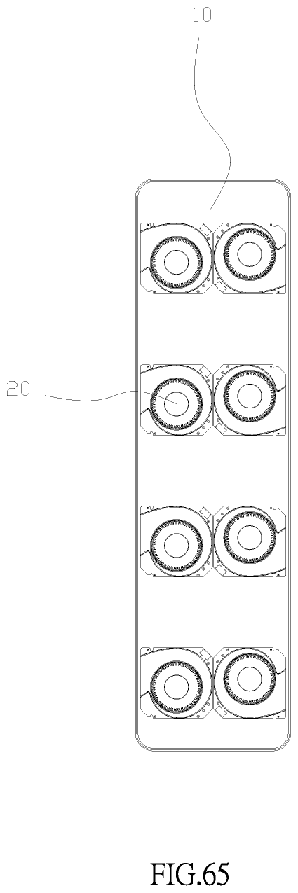

FIG. 66 shows the status diagram for the 64^{th} column of motor modules in this new utility of multi-motor air purifier provided from relatively better cases.

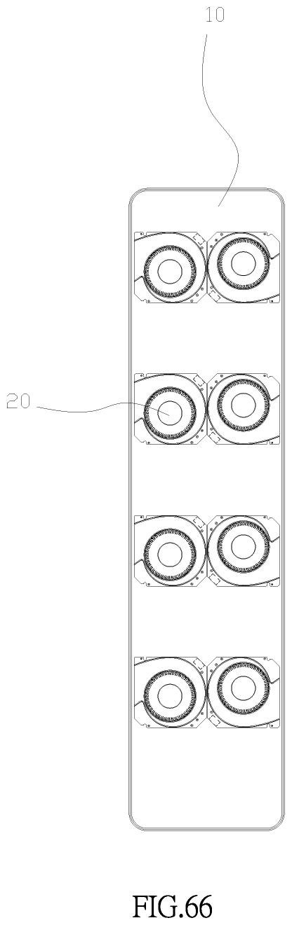

FIG. 67 shows the status diagram for the 65^{th} column of motor modules in this new utility of multi-motor air purifier provided from relatively better cases.

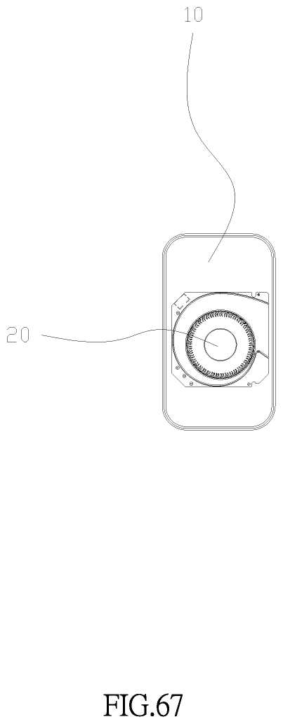

FIG. 68 shows the status diagram for the 66^{th} column of motor modules in this new utility of multi-motor air purifier provided from relatively better cases.

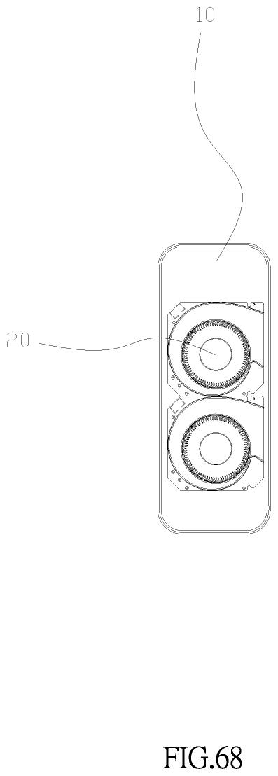

FIG. 69 shows the status diagram for the 67^{th} column of motor modules in this new utility of multi-motor air purifier provided from relatively better cases.

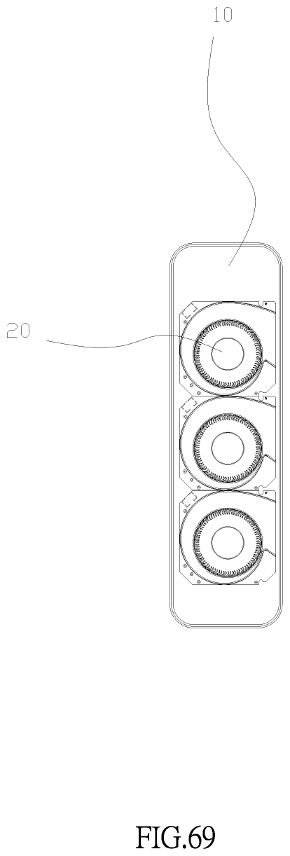

FIG. 70 shows the status diagram for the 68^{th} column of motor modules in this new utility of multi-motor air purifier provided from relatively better cases.

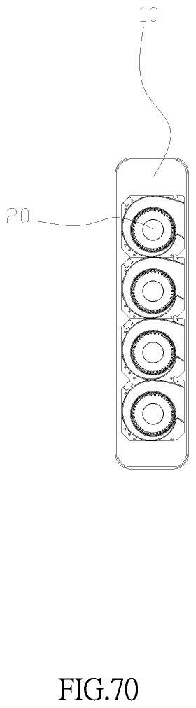

FIG. 71 shows the status diagram for the 69^{th} column of motor modules in this new utility of multi-motor air purifier provided from relatively better cases.

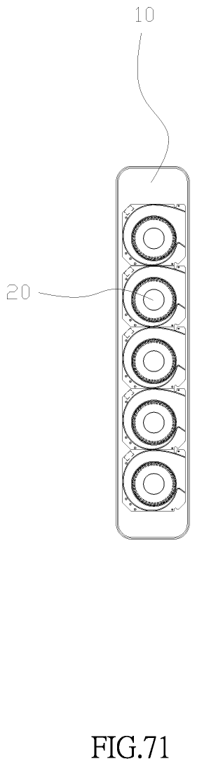

FIG. 72 shows the status diagram for the 70^{th} column of motor modules in this new utility of multi-motor air purifier provided from relatively better cases.

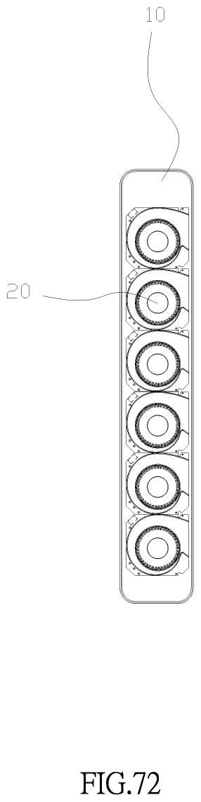

FIG. 73 shows the status diagram for the 71^{st} column of motor modules in this new utility of multi-motor air purifier provided from relatively better cases.

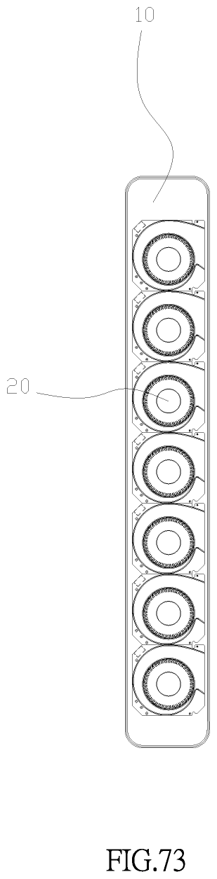

FIG. 74 shows the status diagram for the 72^{nd} column of motor modules in this new utility of multi-motor air purifier provided from relatively better cases.

### SPECIFIC IMPLEMENTATION METHOD

The specific description of the application case for this new utility is as follows. It is shown in the figures that, the same or similar symbol indicates the same or likewise component or components which possess the same or likewise functions.

Except for otherwise stipulated and defined, the terms “assembly”, “to be connected”, “connected” appear in this new utility description are supposed to be comprehended in a broad sense. For instance, “to be connected” may refers to “fixed connection”, “detachable connection”, “integrated connection”; as well as “mechanical connection”, “direct connection”; “connected via mediated approach” or “inner connection between two components”. For the ordinary technicians in this field, they can comprehend the above terms in this new utility as the specific definition in accordance with the actual circumstances.

In view of attached figures in the user manual, the specific implementation method of this new utility is further described to make the technical solution and its beneficial effects much clear. Below descriptions of the application case based on attached figures are examples, which aim to explain this new utility. It cannot be regarded as the limitation for this new utility.

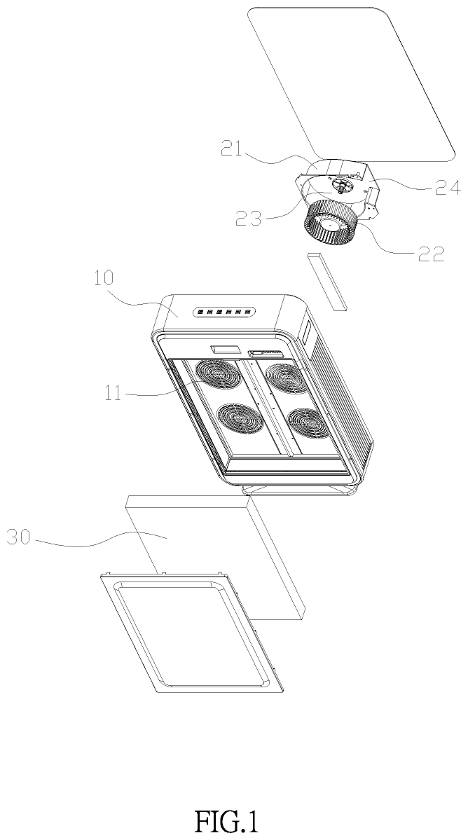

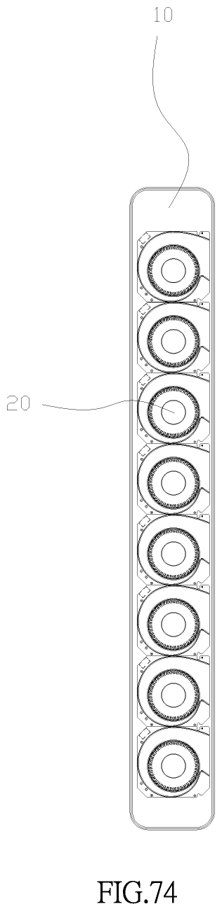

The optimal application cases for this new utility are shown from FIG. 1 to FIG. 74. This multi-motor air purifier comprises chassis No. 10. This chassis possesses the air inlet area No. 11 and the air exhaust area No. 12. The air inlet area No. 11 is installed on the back of the chassis No. 10. And the air exhaust area No. 12 is installed on both sides of the chassis.

The air inlet area No. 11 consists of some motor assembly positions No. 13 which are left for installing the motor module No. 20. Moreover, the motor module No. 20 is evenly arranged in the mentioned air inlet area No. 11 to ensure large-range coverage on the air inlet area. The single-motor module or multi-motor module works together to inhale the airflow from air inlet area to the chassis, then exhaust it out of the air-exhaust area. The motor module No. 20 is arranged in the exhaust area in a horizontal, vertical or circled style.

The traditional air purifier uses single-motor module structure. Consequently, its wind pressure in the core part is extraordinarily large and the wind pressure in the surrounding area is especially small. This results in over-inhalation at the center of filter, which disable the filter to intercept formaldehyde, PM2.5 and other harmful substances. What is worse, the harmful matters flow out of the exhaust outlet again and forms secondary pollution. Simultaneously, the inhaling capacity on the surrounding area of filter is so small that it cannot fully absorb the formaldehyde, PM2.5 and else hazardous matters. In addition, the single-motor module requires high rotating speed to guarantee the required airflow rate. It directly increases noise and peak hour power consumption, which is unfavorable to power conservation and quietness.

This patent technology can solve above mentioned issues as a result of the multi-motor module. This new utility possesses large air-inlet area and air-exhaust area. It decreases the size of motor module to enlarge the coverage of air-inlet area, thus ensuring even distribution of wind pressure.

It also prevents the polarized distribution of wind pressure as well as effectively improves the inhaling capacity and lifespan of the filer by over 35%. This contributes to environmental protection and less usage of consumable materials. Moreover, the rotating speed of multi-motor module is lower than that of single-motor module to achieve the same blowing rate. Therefore, the multi-motor module releases less noise and consumes less peak-hour electric power in comparison to the single-motor module. This is favorable to power conservation and quietness. Furthermore, the multi-point air exhaust area has increased by three times. Compared with the traditional air purifier, the noise of new utility has decreased by 40%. Accordingly, its power consumption has reduced to the lowest level with more than fifteen CADR per watt.

The mentioned motor module No. 20 consists of motor No. 21 and housing No. 22. The mentioned motor No. 21 is installed in the housing No. 22. And the mentioned housing No. 22 possesses air inlet No. 23 and air exhaust outlet No. 24. The inlet No. 23 is connected to the exhaust outlet No. 12 and the exhaust outlet No. 11 is connected to the air exhaust area No. 12, too.

The purifying module No. 30 is placed between the air-inlet area No. 11 and the motor module No. 20. The motor module No. 20 and the purifying module No. 30 are mounted inside the chassis No. 10; In accordance with the purifying method and principle, the purifying module varies accordingly. The purifying media is a filter plate what can screen and intercept the particles in the airflow. The mentioned purifying media is definitely an absorbent body that can produce static electric field and negative ions.

In the description of user manual, the terms “an implementation case”, “preferably”, “example”, “specific example” or “some examples” refers to specific characteristics, structure, material or features in the light of this implementation case or typical examples. It is included in at least one implementation case or example of this new utility. The indicative expression in this user manual does not necessarily refer to the same implementation case or example. Furthermore, the described specific characteristics, structure, materials or features can be combined with together in any implementation cases or examples in a proper way.

According to above description of the structure and principle, the technicians in this field are supposed to comprehend that, this new utility is not limited to the detailed implementation methods. Any widely-known technical improvement and substitution in this field on the basis of this new utility are subject to the protective range of this new utility. It shall be defined as per the requirement of each claim.

What is claimed is:

1. This is a kind of multi-motor air purifier with chassis. Its characteristics is: the mentioned chassis includes both air-inlet area and exhaust area. And the mentioned air-inlet area designed several motor positions which are used for assembling the motor modules. Furthermore, the mentioned motor modules are evenly aligned in above mentioned air-inlet area. The single-motor module and the multi-motor module can work together to bring the air from the air-inlet area into the chassis, then exhaust it out from the air-exhaust area.

2. In according to the multi-motor air purifier as claimed in claim 1, its characteristics is: the motor modules are placed on a horizontal, vertical or circled style in the mentioned air exhaust area.

3. In according to the multi-motor air purifier as claimed in claim 1, its feature is: purifying modules are set between the mentioned air-inlet area and the motor modules. The mentioned purifying module is used to screen the particles in the air and separate them with the filter plate.

4. The characteristics of the multi-motor air purifier as claimed in claim 3, the mentioned purifying module works as the adsorbent body which can produce static electric field.

5. For the multi-motor air purifier as claimed in claim 3, its characteristics is that the mentioned purifying module is a kind of adsorbent body that can produce negative ions.

6. In according to the multi-motor air purifier as claimed in claim 1, its characteristics is: the mentioned air-inlet area is placed on the back of the chassis. The mentioned exhaust area is designed on the side of chassis.

7. In according to the multi-motor air purifier as claimed in claim 3, its characteristics is:
the mentioned motor module and the purifying module are mounted inside the chassis.

8. For the multi-motor air purifier as claimed in claim 1, its characteristics is: the mentioned motor module consists of the motor and housing. The mentioned motors are installed inside the housing. And the housing possesses air inlet and air outlet. The mentioned air inlet is connected to the air exhaust area, and the mentioned air outlet is connected to the air exhaust area as well.
